(19) United States
(12) Patent Application Publication
(10) Pub. No.: US 2006/0205626 A1
(43) Pub. Date: Sep. 14, 2006

(54) Cleaning compound for a medical or dental office

(76) Inventors: Robert Gene Gant, Jersey Village, TX (US); Teresa Leigh Barr, Hood River, OR (US)

Correspondence Address: BUSKOP LAW GROUP, P.C., 1776 YORKTOWN, SUITE 550, HOUSTON, TX, 77056, US

(21) Appl. No.: 11/373,350

(22) Filed: Mar. 10, 2006

Related U.S. Application Data
(60) Provisional application No. 60/660,760, filed on Mar. 11, 2005.

(51) Int. Cl. C11D 3/00

(52) U.S. Cl. 510/367

(57) ABSTRACT

A chemical cleaning composition for medical and dental suction, surgical and evacuation equipment. The chemical cleaning composition comprising a hydroxycarboxylic acid, wherein the hydroxycarboxylic comprises from about 1 weight percent to about 20 weight percent. A peroxymonosulfate compound can be added, wherein the peroxymonosulfate comprises from about 10 weight percent to about 60 weight percent. Sodium tripolyphosphate can be added, wherein the sodium tripolyphosphate comprises from about 10 weight percent to about 30 weight percent. An enzyme can be added to the mixture. Carboxymethylcellulose can be added to the composition, wherein the carboxymethylcellulose comprises from about 1 weight percent to about 2 weight percent.

### CROSS REFERENCE TO RELATED APPLICATIONS

The present application claims priority to co-pending U.S. Provisional Patent Application Ser. No. 60/660,760, filed on Mar. 11, 2005.

### FIELD

The embodiments relate generally to cleaning compounds for use in a medical or dental office.

### BACKGROUND

Solid cleaning, rinsing, and antimicrobial compositions have not been employed in a medical environment, such as for cleaning, rinsing, or antimicrobial treatment of, medical instruments, medical devices, or other medical equipment. The markets for equipment and supplies for washing, medical devices or instruments, or other medical equipment are distinct from markets for cleaning other wares, such as kitchen wares.

A need exists for compositions that are capable of performing a quick and easy cleaning of medical extraction equipment, such as in dentist offices where the suction equipment can remove hard materials such as silver, gold, epoxy or other similar material used in a dentist practice.

Medical devices, such as suction equipment, and other lumened instruments, are subjected to thorough cleaning and antimicrobial decontamination between each use. During medical procedures, the devices become coated with blood and other protein-rich body fluids. If the instruments are cleaned while they are coated with these materials, the high temperatures and/or chemicals used in the cleaning process tend to cause the materials to set as a hardened layer of biological residue that becomes difficult to remove. Not only do such residues present a barrier to cleaning agent penetration, but even when clean, these resdiues may later break down to form toxic substances which pose hazards to patients when the devices are reused.

Traditionally, such devices are often rinsed in a cleaning solution, such as an enzymatic cleaner, to remove the bulk of the blood and other body fluids from their surfaces. The rinsing process is generally carried out manually by immersing the devices in a shallow tray of the cleaning solution. However, for devices such as suction equipment, the cleaning fluid may not penetrate the length of the internal lumen, leaving a portion of the endoscope to become coated with dried body fluids. Additionally, the biological materials and strong cleaners may pose hazards to personnel coming into contact with them.

High temperature cleaning processes, such as steam cleaning in an autoclave, are generally unsuited to the cleaning of suction equipment because of the delicate components and materials from which they are manufactured. The high temperature and pressure tend to curtail the useful life of suction equipment, rubber and plastic devices, lenses, and portions of devices made of polymeric materials and the like. Any body fluids that are not removed prior to thermal cleaning are typically baked on to the instrumentation.

Other types of drain cleaning compositions exists that involve materials different than aluminum for producing the necessary heat for making such a composition effective as a drain cleaner include those involving the reaction between caustic alkali and a substance which is acidic in nature. However, such compositions leave a lot to be desired regarding loss of effectiveness of the active ingredients during storage due to chemical incompatibility of one or more of the ingredients with each other, and additionally resulting from inherent moisture contained in the ingredients.

The embodiments meet these needs.

### DETAILED DESCRIPTION OF THE EMBODIMENTS

Before explaining the embodiments in detail, it is to be understood that the embodiments are not limited to the particular embodiments and that they can be practiced or carried out in various ways.

The embodiments relate generally to the decontamination arts. The embodiments relate to chemical cleaning compositions for cleaning medical and dental: suction, surgical, and evacuation equipment. The embodiments relate to automated systems for leak testing cleaning, and drying devices for medical, dental, mortuary, veterinary and pharmaceutical applications, and the like, and can be described with particular reference thereto. The embodiments are also applicable to the decontamination of other devices in an automated processing system.

The embodied compositions are ecologically safe cleansers useful in all medical and dental fields. The compositions can be used in medical and dental, suction, surgical, and evacuation devices simply by varying the total concentration of the active components in the composition.

The embodied compounds can help preserve the environment by using ingredients that are environmentally friendly and typically used in the food industry as food and supplement additives. The embodied compound can be environmentally friendly because the compounds can be non acidic as well as biodegradable. The embodied compounds can help preserve the environment by eliminating harsh chemicals that have been proven to negatively impact the environment.

The embodied compounds can help save energy by minimizing the electricity needed to clean equipment, as well as human labor. The embodied compounds can help save lives by killing bacteria left on equipment that can transfer hazardous biological waste from patient to patient.

The embodiments of the compound do not require foaming agents. Foaming agents cause a suction system to malfunction due to their inability to be rinsed thoroughly from the equipment. The foam bubbles dry with accumulations of particulate of bacteria and other matter. The pumps of the medical and dental, suction, surgical and evacuation devices comprise close tolerances between moving brass parts. These close tolerances don't allow granular sand, or abrasives to operate in the system as they scratch and damage the pumps.

The embodied compounds do not require detergents. Detergents cause suction systems to malfunction due to the inherent foaming capabilities and the harsh pH of their compounds may damage lumen surfaces. The embodiments of the compound do not change the pH level of the tubing that the compound is moving through. A change in pH level can, degrade the polymer.

The embodiments of the compound can be used for cleaning a variety of devices including garbage disposals, soda fountain tap drains, or a veterinary device with tubing.

In an embodiment, a chemical cleaning composition for medical and dental suction, surgical and evacuation equipment includes hydroxycarboxylic acid, peroxymonosulfate compound, sodium tripolyphosphate, sodium carboxymethylcellulose, a protease enzyme, and an amylase enzyme. The composition is unique because none of the active components of the composition works well alone as a cleanser, but the mixture of the components results in a very effective cleaning composition.

The hydroxycarboxylic acid is an effective chelating agent for mineral salts in the very highly acidic environment of the peroxymonosulfate compound. Examples of hydroxycarboxylic acid usable in the embodied compositions include citric acid and tartaric acid. Citric acid can be known as 2-hydroxy-1,2,3-propanetricarboxylic acid, beta-hydroxytricarballylic acid, aciletten, citretten, citro, hydrocerol A. A molecular formula for citric acid can be HOC(COOH)(CH2COOH)2. The citric acid can appear as a white crystalline powder. Citric acid can comprise a melting point 153 degrees Celsius, but decomposes at the boiling point. Citric acid can comprise a density of 1.54 g cm-3. Citric acid can comprise a flash point of >200 degrees Fahrenheit (93 degrees Celsius), explosion limits, lower 28; upper 2.29, an autoignition temperature of 1010 degrees Celsius. Citric acid can be water soluble. Citric acid is stable, but is incompatible with bases, strong oxidizing agents, reducing agents, and metal nitrates. Citric acid can be easily transported. Citric acid can be transported by air, sea and road freight since the compound is non-hazardous.

Examples of peroxymonosulfate compounds usable in the embodied compositions include potassium peroxymonosulfate, potassium peroxomonosulphate, potassium peroxymonosulphate, potassium hydrogen monopersulfate, potassium hydrogen monopersulphate, and combinations thereof.

Potassium peroxymonosulfate is potassium monopersulfate, which is present as a component of a triple salt with a formula of: 2KHSO5.KHSO4.K2SO4. The oxidation potential of potassium peroxymonosulfate is derived from the compound' peracid chemistry. Potassium peroxymonosulfate is the first neutralization salt of peroxymonosulfuric acid and can have a formula of H2SO5. Potassium peroxymonosulfate is highly and readily soluble in water as shown in Table III. At 20° C. (68° F.), the solubility of Potassium peroxymonosulfate in water is >250 g/L.

The standard electrode potential (Eo) of potassium peroxymonosulfate is shown in the following reaction: HSO4−+H2O→HSO5−+2 H++2 e− −1.44 V. This standard electrode potential is high enough for many room temperature oxidations, including halide to halogen or hypohalite, ferrous ion to ferric, and manganous ion to manganic.

At concentrations above saturation, potassium sulfate can precipitate, but additional potassium monopersulfate can remain in a solution, so that the attainable % active oxygen in the solution is higher than is indicated in Table I. Solutions of Potassium peroxymonosulfate are relatively stable when made up at the unmodified pH of the product. The stability is adversely affected by higher pH, especially above pH 7. A point of minimum stability exists at pH 9, at which the concentration of the mono-anion HSO5−is equal to that of the di-anion SO5=. Iron, cobalt, nickel, copper, manganese, and other transition metal ions can catalyze the decomposition of Potassium peroxymonosulfate in the solution; the degree to which catalysis occurs is dependent on the concentrations of potassium peroxymonosulfate and of the metal ion.

TABLE I
Solubility of Potassium peroxymonosulfate or monopersulfate in Water

| | |
|---|---|
| Temperature, | Active Oxygen |

| ° C. | ° F. | Solubility, g/L | In Solution, wt % |
|---|---|---|---|
| 20 | 68 | 256 | 0.92 |
| 27 | 80 | 268 | 0.95 |
| 49 | 120 | 300 | 1.04 |
| 60 | 140 | 315 | 1.08 |
| 71 | 160 | 335 | 1.13 |

A specific example of a peroxymonosulfate compound is Oxone®. Oxone® is available from E. I. duPont de Nemours Company. Oxone® contains the active ingredient potassium peroxymonosulfate. The physical properties and typical analyses of Oxone® are shown in Table II.

TABLE II

| | | |
|---|---|---|
| Molecular Weight | (triple salt) | 614.7 |
| Active Oxygen | % min. | 4.5 |
| | % average analysis | 4.7 |
| | % theoretical (triple salt) | 5.2 |
| Active Component | % KHSO₅, min. | 42.8 |
| Bulk Density | g/cm³ (Mg/m³) | 1.15-1.30 |
| | lb/ft³ | 72-81 |
| Particle Size | through USS Sieve #20, % | 100 |
| | through US Sieve #200, % min. | 10 |
| | (also see Table III) | |
| pH | at 25° C. (77° F.) | |
| | 1% solution | 2.3 |
| | 3% solution | 2.0 |
| Solubility | g/L, H₂O, 20° C. (68° F.) | 256 |
| | (also see Table I) | |
| Moisture Content | % max. | 0.1 |
| Stability | % active oxygen loss/month | <1 |
| Standard Electrode | V | −1.44 |
| Potential (E°) | | |
| Heat of Decomposition | kJ/kg | 251 |
| | Btu/lb | 108 |

Oxone® is a relatively stable peroxygen, and loses less than 1% of its activity per month when stored under appropriate conditions. However, like all other peroxygens, Potassium peroxymonosulfate undergoes very slow decomposition in storage, with liberation of oxygen gas and a small amount of heat. Decomposition of Potassium peroxymonosulfate generates oxygen gas. If a decomposition is associated with high temperature, decomposition of the constituent salts of Potassium peroxymonosulfate may generate sulfuric acid, sulfur dioxide, or sulfur trioxide. The stability is reduced by the presence of small amounts of moisture, alkaline chemicals, chemicals which contain water of hydration, transition metals in any form, and/or any material with which Potassium peroxymonosulfate can react. The decomposition of Potassium peroxymonosulfate is exothermic; this property can cause the decomposition to accelerate if conditions allow the product temperature to rise.

A particle size analysis of Oxone® is shown in Table II. Particle size can be adjusted by screening, grinding, or compaction/granulation processes. The product temperature can be kept below 50° C. (122° F.) at all times during such operations; packaging temperature can not exceed 30° C. (86° F.).

TABLE III
Typical Particle Size Analysis of Potassium
peroxymonosulfate Monopersulfate

| U.S. Sieve Size | Sieve Opening, μm | Approx. Composition, wt % |
|---|---|---|
| 30 | 600 | 1 |
| 70 | 212 | 68 |
| 100 | 150 | 84 |
| 200 | 75 | 98 |
| 325 | 45 | 100 |

Sodium tripolyphosphate can have an appearance of white or colorless crystals, granules or powder. Sodium tripolyphosphate can comprise a melting point of 622 degrees Celcius, and a boiling point of more than 1000 degrees Celsius. Sodium tripolyphosphate also can comprise a specific gravity of from about 1.1 to about 1.4. Examples of sodium tripolyphosphate usable in the embodied compositions include triphosphoric acid pentasodium salt and pentasodium triphosphate. The molecular formula for sodium tripolyphosphate can be: Na5O10P3.

Sodium carboxymethylcellulose or the sodium salt of carboxymethylcellulose can be a water-soluble component created from naturally occurring cellulose by means of grafting small anionic groups onto the cellulose backbone. Since the cellulose is partially modified, sodium carboxymethylcellulose can be more bio-friendly than synthetic ingredients, since cellulose enzymes found in the environment are still able to break down the unmodified portions of the sodium carboxymethylcellulose molecule. The carboxymethlcellulose can be a non-foaming suspension agent. The carboxymethlcellulose acts as a buffer to prevent breakdown in water, and the excess heat caused by sodium hydroxide. A molecular formula for sodium carboxymethylcellulose can be: NaCMC.

Sodium carboxymethylcellulose can be a hygroscopic powder containing very fine granules, particles or fibers and can be white to slightly yellow in color and can be practically odorless and tasteless. Sodium carboxymethylcellulose can be used in concentrations from about 1 weight percent to about 2 weight percent to give good suspending properties. Sodium carboxymethylcellulose is easily dispersed in water forming a colloidal solution. Sodium carboxymethylcellulose exhibits maximum viscosity at pH 7-9, and some viscosity is reduced below pH 4 and above pH 10. The carboxymethylcellulose can cause the composition to disperse by giving more substance to the composition. The carboxymethylcellulose can also cause a cat ionic bond to the particles that are being removed from the item being cleaned, causing the particles to be removed as the composition passes by the particle.

Sodium carboxymethylcellulose can be a NF/Super disintegrant used in dietary supplements and can also be used as a thickener, binder and emulsifying agent in food and cosmetics. Sodium carboxymethylcellulose can be obtained from Hercules Incorporated, of Wilmington, Del.

The embodied compositions can include enzymes. Enzymes are proteins, which catalyze reactions within the cleansing cycle. Enzymes can lower the operating temperature, and shorter cleaning cycles are some of the advantages of using enzymes in the composition. An enzyme can be a non-surfactant. The pH of the composition can remain the same with the addition of enzymes. Enzymes can have a negative charge, which can attract the iron in blood which is positively charged. The remaining negatively charged particles that are to be removed are attracted to the cat ionic charge of the carboxymethylcellulose.

Examples of enzymes usable in the embodied compositions include protease and amylase. Protease enzymes are necessary to break down and remove protein-based particles such as blood products, such as hemoglobin.

The bacterial alkaline protease (E.C.3.4.21.14) can be produced by the controlled fermentation of Bacillus licheniformis, which can be an endopeptidase capable of hydrolyzing the interior peptide bonds of protein molecules. The broad substrate specificity enables the enzyme to effectively hydrolyze most proteins. Hemoglobin, casein, egg yolk, soya, gelatin, fish and other proteins are hydrolyzed to lower molecular weight peptides.

The bacterial alpha-amylase (3-2.1, 1) can originate from an endoamylase capable of randomly hydrolyzing the alpha-1, 4-glucosidic linkages of starch. The enzyme hydrolyzes starch, amylose and amylopectin to soluble dextrins and small quantities of glucose and maltose. Amylase enzymes are necessary to break down starch based products such as plaque and tartar or even foods such as gravy or chocolate.

Enzymes such as lipase, cellulase, amylase and protease are commonly used in addition to viable bacterial products to eliminate waste items in grease trap cleaners, septic system maintenance products and wastewater treatment formulas.

A specific example of a peroxymonosulfate compound is Deterzyme PAG 520/220 and distributed by Deerland Corporation of Kennesaw, Ga. Deterzyme PAG 520/220 is a blend of protease and amylase enzymes. Deterzyne PAG 520/220 is completely water soluble and is compatible with most detergents and components, and can be stable in dilute solutions at temperatures up to 50 degrees Celsius. Deterzyne PAG 520/220 is available in sealed 50 Kg polyethylene fiber drums and is very stable with a loss of activity of no more than 10 percent over six months. Storing under refrigeration at 5 degrees Celsius can extend storage life.

In an example embodiment, the embodied chemical cleaning compositions include from about 1 weight percent to about 20 weight percent of hydroxycarboxylic acid; from about 10 weight percent to about 60 weight percent of peroxymonosulfate compound; from about 10 weight percent to about 30 weight percent of sodium tripolyphosphate; from about 0.01 weight percent to about 20 weight percent of sodium carboxymethylcellulose; from about 0.01 weight percent to about 10 weight percent of a protease enzyme; and from about 0.01 weight percent to about 10 weight percent of an amylase enzyme.

In an example embodiment, the embodied chemical cleaning compositions include hydroxycarboxylic acid, potassium peroxymonosulfate and tripolyphosphate. The embodied compound has the properties of a non-foaming cleaning solution and cleaned evacuation equipment during a test.

In an example embodiment, the embodied chemical cleaning compositions include hydroxycarboxylic acid, potassium peroxymonosulfate, tripolyphosphate and sodium carboxymethylcellulose. The embodied compound has the properties of a non-foaming cleaning solution and cleansed as well as carried away more debris and particles during tests of the composition as compared to a composition without carboxymethylcellulose.

In an example embodiment, the embodied chemical cleaning compositions include hydroxycarboxylic acid, potassium peroxymonosulfate, tripolyphosphate, sodium carboxymethylcellulose and a protease and amylase enzyme blend. The embodied compound has the properties of a non-foaming cleaning solution and carried away debris and particles, additionally the enzymes biodegraded proteins and starches during tests of a composition comprising enzymes.

The embodied compositions can be stored in a water tight container because decomposition can be accelerated with contact to moisture. The embodied compositions can be granular without any water content. The lack of water content prevents degrading of the compound during storage. The embodied compositions can be stored in a water-resistant liner, but storage conditions can also include provisions for prevention of contact with water, including high airborne humidity.

An embodied method for cleaning a dental evacuation device includes mixing a formulation of a hydroxycarboxylic acid, a peroxymonosulfate compound and a sodium tripolyphosphate. Water is added to the formulation to create a cleaning solution. The dental evacuation devices can then be submerged in the formed solution. The formed solution can be run through the evacuation equipment, wherein after the solution has run through the evacuation equipment, the evacuation equipment is cleared of all obstructions and should not be damaged in any way.

The embodiments of the compound can be used in a 3450 rpm pump system or any similar evacuation system. These pump systems can be used to extract fillings and pull the filings through a 1 inch ID suction line. The compound can break down the fillings, blood tissue, and other items that can clog the suction line of a medical or dental, suction, surgical or evacuation device.

While these embodiments have been described with emphasis on the embodiments, it should be understood that within the scope of the appended claims, the embodiments might be practiced other than as specifically described herein.

What is claimed is:

1. A chemical cleaning composition comprising:
(a) from about 1 weight percent to about 20 weight percent of a hydroxycarboxylic acid;
(b) from about 10 weight percent to about 60 weight percent of a peroxymonosulfate compound;
(c) from about 10 weight percent to about 30 weight percent of sodium tripolyphosphate; and
(d) an enzyme.

2. The chemical cleaning composition of claim 1, further comprising from about 1 weight percent to about 2 weight percent of carboxymethylcellulose.

3. The chemical cleaning composition of claim 1, further comprising a starch.

4. The chemical cleaning composition of claim 1, wherein the hydroxycarboxylic acid is selected from the group consisting of citric acid, sodium sulfate, sodium bisulfate, tartaric acid, and combinations thereof.

5. The chemical cleaning composition of claim 1, wherein the peroxymonosulfate compound is selected from the group consisting of potassium peroxymonosulfate, potassium peroxomonosulphate, potassium peroxymonosulphate, potassium hydrogen monopersulfate, potassium hydrogen monopersulphate, and combinations thereof.

6. The chemical cleaning composition of claim 1, wherein the enzyme is selected from the group consisting of protease, amalyse, and combinations thereof.

7. The chemical cleaning composition of claim 6, wherein the amalyse comprises from about 0.01 weight percent to about 10 weight percent.

8. The chemical cleaning composition of claim 6, wherein the protease comprises from about 0.01 weight percent to about 10 weight percent.

9. A chemical cleaning composition comprising:
(a) from about 1 weight percent to about 20 weight percent of a hydroxycarboxylic acid;
(b) from about 10 weight percent to about 60 weight percent of a peroxymonosulfate compound;
(c) from about 10 weight percent to about 30 weight percent of sodium tripolyphosphate; and
(d) from about 1 weight percent to about 2 weight percent of carboxymethylcellulose.

10. The chemical cleaning composition of claim 9, further comprising an enzyme, wherein the enzyme is selected from the group consisting of protease, amalyse, and combinations thereof.

11. The chemical cleaning composition of claim 10, wherein the amalyse comprises from about 0.01 weight percent to about 10 weight percent.

12. The chemical cleaning composition of claim 10, wherein the protease comprises from about 0.01 weight percent to about 10 weight percent.

13. The chemical cleaning composition of claim 9, further comprising a starch.

14. The chemical cleaning composition of claim 9, wherein the hydroxycarboxylic acid is selected from the group consisting of citric acid, sodium sulfate, sodium bisulfate, tartaric acid, and combinations thereof.

15. The chemical cleaning composition of claim 9, wherein the peroxymonosulfate compound is selected from the group consisting of potassium peroxymonosulfate, potassium peroxomonosulphate, potassium peroxymonosulphate, potassium hydrogen monopersulfate, potassium hydrogen monopersulphate, and combinations thereof.
